(19) United States
(12) Patent Application Publication
(10) Pub. No.: US 2021/0393799 A1
(43) Pub. Date: Dec. 23, 2021

(54) DENDRITIC POLYMERS COMPLEXED WITH IMMUNE CHECKPOINT INHIBITORS FOR ENHANCED CANCER IMMUNOTHERAPY

(71) Applicant: Wisconsin Alumni Research Foundation, Madison, WI (US)

(72) Inventors: Seungpyo Hong, Madison, WI (US); Jiyoon Bu, Madison, WI (US)

(21) Appl. No.: 17/287,748

(22) PCT Filed: Oct. 29, 2019
(86) PCT No.: PCT/US2019/058463

§ 371 Date: Apr. 22, 2021

Related U.S. Application Data
(60) Provisional application No. 62/751,831, filed on Oct. 29, 2018.

(51) Int. Cl. A61K 47/69; A61K 47/59

(52) U.S. Cl. CPC A61K 47/6935; A61K 47/595

(57) ABSTRACT

Described herein is a nanoparticle system including a multivalent nanoparticle core having a plurality of immune checkpoint inhibitors conjugated thereto. Also included are pharmaceutical compositions and methods of making the nanoparticle system. Further included are immunotherapy methods including administering the nanoparticle system to a subject in need thereof, such as a human cancer patient.

### CROSS-REFERENCE TO RELATED APPLICATIONS

This application claims priority to U.S. Provisional Application 62/751,831 filed on Oct. 29, 2018, which is incorporated herein by reference in its entirety.

### FIELD OF THE DISCLOSURE

The present disclosure is related to compositions and methods for cancer immunotherapy with immune checkpoint inhibitors.

### BACKGROUND

Tumor cells have immune escape mechanisms by triggering immune checkpoint regulators, such as PD-1/PD-L1 or CTLA-4/B7. These interactions exhibit immune-inhibitory behaviors, causing apoptosis of cytotoxic T lymphocytes, suppressing release of cytokine signaling molecules in the immune system, and increasing immune dysfunction. These results collectively contribute to the intratumoral microvessel formation and higher chemoresistance of the tumor cells. Therefore, inhibition of immune checkpoint regulators can restore antigen-specific T cells and suppress tumor proliferation.

Immune checkpoint inhibition can be achieved by targeting either T cells via blocking receptors such as CTLA-4 and PD-1, or cancer cells via blocking proteins such as PD-L1 and PD-L2. PD-1 and PD-L1 are targets for cancer immunotherapy, for example, because the blockade of their interaction halts or limits T cell response and results in the reactivation of anticancer immunity and, in turn, tumor regression. Several monoclonal antibodies, peptides, proteins, and other small molecules have been developed to target immune checkpoint regulators, such as pembrolizumab and novilumab for targeting PD-1 and atezolizumab, avelumab, and durvalumab for PD-L1. However, recently published clinical results of such immune checkpoint inhibitors (ICIs) molecules have demonstrated poor clinical outcomes where low response rates were reported from various cohorts.

What is needed are new compositions and methods for cancer immunotherapy using ICIs.

### BRIEF SUMMARY

In one aspect, a nanoparticle system comprises a multivalent nanoparticle core comprising a plurality of immune checkpoint inhibitors conjugated thereto.

In another aspect, pharmaceutical composition comprises the nanoparticle system and a pharmaceutically acceptable excipient.

In yet another aspect, a method of making a nanoparticle system comprises contacting multivalent nanoparticle cores comprising multiple reactive end groups with a composition comprising one or more immune checkpoint inhibitors under conditions sufficient to conjugate a plurality of the immune checkpoint inhibitors to the multivalent nanoparticle cores and provide the nanoparticle system.

In another aspect, an immunotherapy method comprises administering the nanoparticle system to a subject in need thereof.

### BRIEF DESCRIPTION OF THE DRAWINGS

FIG. 1 is a schematic illustration depicting the hypothesis of enhanced cancer immunotherapy via dendrimer-meditated multivalent binding effect. The enhanced binding kinetics between the G7-aPD-L1 conjugates and target receptor (PD-L1) results in improved inhibition of the PD-1/PD-L1 interaction, improving immunotherapy efficacy.

FIG. 2 illustrates the synthesis of generation 7(G7) poly(amidoamine)(PAMAM) dendrimer and anti-PD-L1 antibody conjugates (G7-aPD-L1). G7 PAMAM dendrimers were labelled with Alexa Fluor® 647, followed by partial acetylation using acetic anhydride. The remaining amine terminal groups were then carboxylated with succinic anhydride. The carboxyl end groups on the dendrimers were activated using the EDC/NHS (1-ethyl-3-(3-dimethylaminopropyl)carbodiimide/N-hydroxysuccinimide) chemistry and conjugated with aPD-L1 antibodies at an 1:5 molar ratio. The final conjugates were filtered against a 100k centrifugation filter (10 min for each of three times). The number of antibodies conjugated per dendrimer molecule was measured using a BCA assay. Approximately 3.9±0.6 antibodies were conjugated to each dendrimer.

FIG. 3 illustrates the characterization of the dendrimer conjugates using atomic force microscopy (AFM), confirming the successful conjugation between G7 dendrimers and antibodies. AFM images demonstrate a significant increase in both lateral diameter (D) and height (h) of the G7-Ab conjugates (D=27.4±8.9 nm/h=9.8±3.9 Å) compared to free antibodies (D=12.7±4.4 nm; p<0.001/h=6.7±2.5 Å; p<0.001) and G7 PAMAM dendrimers (D=16.3±7.3 nm; p<0.001/h=5.6±1.6 Å; p<0.001).

FIG. 4 shows the quantification of the AFM characterization of FIG. 3.

FIG. 5A-D show the enhanced binding kinetics of G7-aPD-L1 conjugates were confirmed using (A) surface plasmon resonance (SPR), (B) biolayer interferometry (BLI), and (C) atomic force microscopy (AFM). FIGS. 5A and B show G7-aPD-L1 conjugates exhibited up to two orders of magnitude lower dissociation constant (K_{D}) compared to free aPD-L1. FIG. 5C shows G7-aPD-L1 conjugates tended to show higher rupture force with multiple rupture events compared to aPD-L1 as shown (Left). Histogram of rupture forces as different loading rates were fitted into double Gaussian model (Middle). These were translated into Bell-Evans model to obtain dissociation rate (Right). G7-aPD-L1 conjugates demonstrated an order of magnitude enhanced off-rate kinetics compared to aPD-L1. FIG. 5D shows in summary, G7-aPD-L1 exhibited significantly higher binding kinetics than aPD-L1.

FIG. 6 (left panel) shows PD-L1 expressions of 786-0 (PD-L1^{High}) and MCF-7 (PD-L1^{LOW}) cell lines was quantified by western blot. The right panel shows expressions of both aPD-L1 and G7-aPD-L1 were significantly higher in 786-0 cell line compared to MCF-7.

FIG. 7 shows (left panel) cancer cells were suspended on the surface functionalized with either G7-aPD-L1 conjugates or aPD-L1. The right panel shows PD-L1^{High} cancer cells showed 1.4-fold (p<0.05) enhanced retention on the surface covered with the G7-aPD-L1 conjugates at a shear rate of 25 s⁻¹, compared to that with free antibodies.

FIG. 8 shows a schematic for enhanced blockade of PD-1/PD-L1 interaction via G7-aPD-L1 conjugates in vitro by assessing Jurkat T cell production of IL-2.

FIG. 9 shows that for the assay of FIG. 8, the blockade of the PD-1/PD-L1 pathway via G7-aPD-L1 resulted in 1.9-fold enhancement in T cell IL-2 production (p=0.036).

FIG. 10 shows a schematic for enhanced blockade of PD-1/PD-L1 interaction via G7-aPD-L1 conjugates in vitro by measuring chemo-sensitivity.

FIG. 11 shows that for the assay of FIG. 10, the blockade of the PD-1/PD-L1 pathway via G7-aPD-L1 resulted in 9% reduction in chemoresistance of 786-0 cells to doxorubicin (p=0.002), compared to non-ICI treated cells.

FIG. 12 shows the target specificity of G7-aPD-L1 using mouse oral squamous cell carcinoma (OSCC) cell line, MOC1 (PD-L1^{High}). The upper panel demonstrates that both fluorophore-labelled aPD-L1 and G7-aPD-L1 were highly expressed in MOC1 cells at the concentration of 67 nM. However, the expressions of both inhibitors were significantly reduced when the PD-L1 ligands were blocked by pre-treating the cells with 670 nM of nonfluorescent aPD-L1. Collectively, these results support that G7-aPD-L1 has high selectivity towards PD-L1 protein.

FIG. 13 shows enhanced targeting of G7-aPD-L1 using in vivo mouse model. Experiments were conducted using 4- to 6-week-old female C57BL/6 mice which were obtained from the Envigo Laboratories (Indianapolis, Ind.). All animal procedures and maintenance were conducted in accordance with the institutional guidelines of the University of Wisconsin. To establish an in vivo mouse tumor model, approximately 5×10⁵ MOC1 cells were injected to the mice. Once the tumor reached 300 mm³, 50 μL of either G7-aPD-L1 or aPD-L1 was injected through the tail vein of the tumor-bearing mouse at the concentration of 128 nM. In vivo imaging system (IVIS) analysis reveals approximately 2-fold enhancement of G7-aPD-L1 for targeting the tumor, compared to aPD-L1.

The above-described and other features will be appreciated and understood by those skilled in the art from the following detailed description, drawings, and appended claims.

### DETAILED DESCRIPTION

Described herein is a novel nanoparticle system that effectively inhibits immune checkpoints, the system based on multivalent binding mediated by multibranched polymers. Various monoclonal antibodies, peptides, proteins, and small molecules have been introduced as immunotherapy checkpoint inhibitors (ICIs) and applied in clinical settings. However, recently published clinical results of such inhibitors have shown inconsistent benefit. The compositions and methods described herein significantly improve the efficacy of the ICIs. The inhibition of these checkpoints could be significantly enhanced by employing the multivalent binding effect of hyperbranched polymers, dendrimers, dendrons, and micelles. The conjugates between the dendritic polymers and ICIs enable multivalent inhibition that provides enhanced selectivity, high sensitivity, and strong binding affinity towards the target receptor. Thus, the conjugates can substantially increase overall binding strength and improve the regulation of the immune system process, ultimately enhancing cancer immunotherapy. As used herein, immunotherapy is the use of an individual's own immune system to treat disease, or the use of immune system components to treat disease.

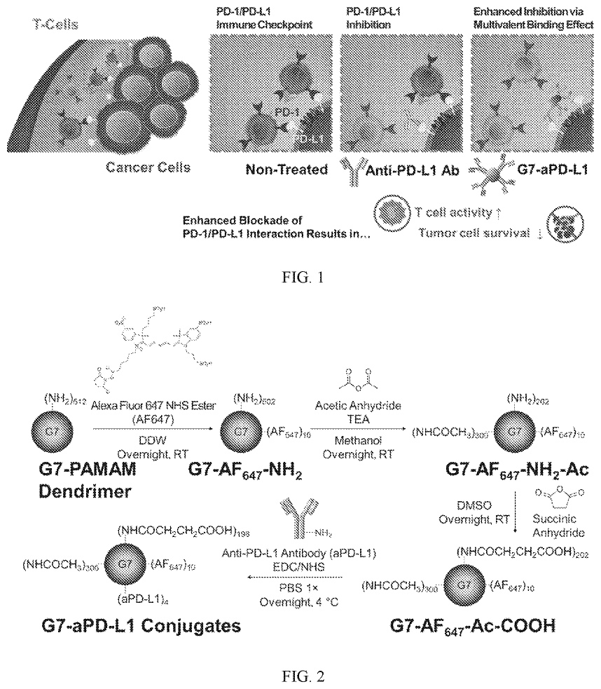

Without being held to theory, it is believed that multivalent, e.g., multibranched, nanoparticles conjugated to multiple ICIs will enhance cancer immunotherapy. ICIs conjugated to a dendritic polymer, for example, are predicted to create stronger binding between the ligand and receptors by forming multiple binding pairs, which is also known as the multivalent binding effect. The multivalent binding effect increases both the intensity and duration of intracellular immune system signaling, which can enhance the inhibition of immune checkpoints. FIG. 1 illustrates an embodiment of the present disclosure in which a PD-L1 antibody-conjugated dendrimer can inhibit the PD-1/PD-L1 interaction more efficiently via multivalent binding.

Advantages of the nanoparticle system described herein include the use of nanoparticulate carriers with high water solubility, biocompatibility, modifiable surface groups, and multivalency.

In an embodiment, a nanoparticle system comprises a multivalent nanoparticle core comprising a plurality of immune checkpoint inhibitors conjugated thereto. The plurality of ICIs can include multiples of the same ICI, or different ICIs conjugated to the same nanoparticle core. In specific embodiments, the multivalent nanoparticle core comprises a hyperbranched polymer, a dendrimer, a dendron, a hybrid nanoparticle, or a micelle. The multivalent nanoparticle cores can have diameters of 3 to 150 nm, for example.

As used herein, hyperbranched polymers are multivalent particles that are polydisperse and irregular in terms of their branching and structure. Dendrimers, in contrast, have a very regular, radially symmetric generation structure. Dendrimers are monodisperse globular polymers which, by comparison with hyperbranched polymers, are typically prepared in multistep syntheses. The dendrimer structure is characterized by the polyfunctional core which represents the center of symmetry, various well-defined radially symmetric layers of a repeating unit (generation) and the terminal groups.

Hyperbranched polymers include polyesters, polyesteramides, polyethers, polyamides, polyethyleneimines, polyglycerols, polyglycolides, polylactides, polylactide-co-glycolides, polytartrates and polysaccharides. Hyperbranched polyesters include Boltorn® from Perstorp AB, hyperbranched polyesteramides include Hybrane® from DSM BV Niederlande, polyglycerols are produced by Hyperpolymers GmbH, and hyperbranched polyethyleneimines include Polyimin® from BASF AG.

Hyperbranched polymers also include polycaprolactones and copolymers such as poly(D,L-lactide-co-glycolides) and the polyester compounds produced by Degussa AG from the Dynapo;®S and Dynacoll® product families.

Preparation of hyperbranched polymers, e.g., hyperbranched polyglycerols, is well known in the art. For example, controlled anionic ring-opening multibranching polymerization of glycidol is performed to form hyperbranched polyglycerols.

Hyperbranched polyglycerols are then reacted with succinic anhydride in pyridine to provide carboxylic acid terminal groups via an ester linkage. Once the functional group content on hyperbranched polyglycerols is verified, the hydroxyl can be further functionalized by the following scheme: hyperbranched polyglycerols-OH+N-(p-maleimidophenyl)isocyanate (PMPI, 10-fold molar excess) in DMSO or DMF at pH 8.5 to obtain hyperbranched polyglycerols-maleimide. Hyperbranched polyglycerols thus possess both carboxyl and maleimide functional groups that can react with corresponding cross-linkers and chemical groups, or can be further derivatized to suit specific functional groups available.

Amphiphilic hyperbranched polymers can form micelle-like structures. The hyperbranched polymer can be an “imperfect” molecule, in that it may include linear sections, and may feature random or unsymmetrical branching. Hyperbranched polymers can be selectively modified to achieve multiple functionalities on the surface and linked to functional components such as carbon chains to install hydrophobicity, and primary amine groups for hydrophilicity and activation for subsequent modifications.

The advantages of hyperbranched polymers include smaller unit sizes (typically <60 nm in diameter) and relatively simple procedures for synthesis. Potential disadvantages include broad size distributions and potential difficulties controlling surface modification for specific functionalities.

The term “dendrimer” as used herein includes, but is not limited to, a molecular architecture with an interior core, interior layers (or “generations”) of repeating units regularly attached to and extending from this initiator core, each layer having one or more branching points, and an exterior surface of terminal groups attached to the outermost generation. Dendrimers have regular dendrimeric or “starburst” molecular structures. Nanoparticle dendrimers generally have diameters of 3 to 10 nm, for example.

Each successive dendrimer generation can be covalently bound to the previous generation. The number of reactive groups of the core structure determines n-directionality and defines the number of structures that can be attached to form the next generation.

The number of branches in a dendritic structure is dependent on the branching valency of the monomeric building blocks, including the core. For example, if the core is a primary amine, the amine nitrogen would then be divalent, resulting in a 1-2 branching motif.

Exemplary dendrimers are alkylated dendrimers such as poly(amido-amine) (PAMAM), poly(ethyleneimine) (PEI), polypropyleneimine (PPI), diaminobutane amine polypropylenimine tetramine (DAB-Am 4), polypropylamine (POPAM), polylysine, polyester, iptycene, aliphatic poly(ether), aromatic polyether dendrimers, or a combination comprising one or more of the foregoing.

The dendrimers can have carboxylic, amine and hydroxyl terminations and can be of any generation including, but not limited to, generation 1 dendrimers (G1), generation 2 dendrimers (G2), generation 3 dendrimers (G3), generation 4 dendrimers (G4), generation 5 dendrimers (G5), generation 6 dendrimers (G6), generation 7 dendrimers (G7), generation 8 dendrimers (G8), generation 9 dendrimers (G9), or generation 10 dendrimers (G10).

The PAMAM dendrimers contain internal amide bonds which may enhance their biodegradability, thus improving tolerance in terms of human therapeutic applications. The surface includes polar, highly reactive primary amine groups. The surfaces of the amino-functional PAMAM dendrimers are cationic and can be derivatized, either through ionic interactions with negatively charged molecules, or using many well-known reagents for covalent functionalization of primary amines.

When PAMAM dendrimers are employed, generations from 0 to 7 PAMAM dendrimers are typically used. For example, hybrid nanoparticles can be formed from generation 0 PAMAM dendrimers (G0); generation 1 (G1) PAMAM dendrimers; generation 2 (G2) PAMAM dendrimers; generation 3(G3) PAMAM dendrimers; generation 4 (G4) PAMAM dendrimers; generation 5 (G5) PAMAM dendrimers; generation 6 (G6) PAMAM dendrimers; or generation 7 (G7) PAMAM dendrimers. PAMAM is commercially available from multiple sources, including Sigma-Aldrich (Cat. No. 597309).

Diaminobutane amine polypropylenimine tetramine (DAB Am 4) is a polymer with a 1,4-diaminobutane core (4-carbon core) with 4 surface primary amino groups. When hybrid nanoparticles are formed from DAB-AM 4 dendrimers, generations from 0 to 7 DAB-AM 4 dendrimers are typically used. For example, hybrid nanoparticles can be formed from generation 0 DAB-AM 4 dendrimers (G0); generation 1 (G1) DAB-AM 4 dendrimers; generation 2 (G2) DAB-AM 4 dendrimers; generation 3(G3) DAB-AM 4 dendrimers; generation 4 (G4) DAB-AM 4 dendrimers; generation 5 (G5) DAB-AM 4 dendrimers; generation 6 (G6) DAB-AM 4 dendrimers; or generation 7 (G7) DAB-AM 4 dendrimers. DAB-Am 4 is commercially available from multiple sources, including Sigma-Aldrich (Cat. No. 460699).

The multivalent nanoparticles may be formed of one or more different dendrimers. Each dendrimer of the dendrimer complex may be of similar or different chemical nature than the other dendrimers (e.g., the first dendrimer can be a PAMAM dendrimer, while the second dendrimer can in be a POPAM dendrimer).

Dendrons are monodisperse, wedge-shaped dendrimer sections with multiple terminal groups and a single reactive function at the focal point. Dendrons can be grafted to a surface, another dendron, or a macromolecule, for example. Bis-MPA (bis-dimethylolpropionic acid) dendrons are available from Sigma-Aldrich.

As used herein, a “micelle” refers to an aggregate of amphiphilic molecules in an aqueous medium, having an interior core and an exterior surface, wherein the amphiphilic molecules are predominantly oriented with their hydrophobic portions forming the core and hydrophilic portions forming the exterior surface. Various monoclonal antibodies, peptides, proteins, and small molecules can covalently bind to the hydrophilic head group of micelles, covering the nanoparticle with plurality of conjugated ICIs for stronger binding kinetics. Micelles are typically in a dynamic equilibrium with the amphiphilic molecules or ions from which they are formed existing in solution in a non-aggregated form. Many amphiphilic compounds, including in particular detergents, surfactants, amphiphilic polymers, lipopolymers (such as PEG-lipids), bile salts, single-chain phospholipids and other single-chain amphiphiles, and amphipathic pharmaceutical compounds are known to spontaneously form micelles in aqueous media above certain concentration, known as critical micellization concentration, or CMC. The amphipathic, e.g., lipid, components of a micelle do not form bilayer phases, nonbilayer mesophases, isotropic liquid phases or solid amorphous or crystalline phases. The concept of a micelle, as well as the methods and conditions for their formation, are well known to skilled in the art. Micelles can co-exist in solution with lipidic particles.

Exemplary micelles include those described in U.S. Pat. No. 9,212,258, incorporated by reference for its disclosure of micelles comprising amphiphilic dendron-coils. Each amphiphilic dendron-coil comprises a non-peptidyl, hydrophobic core-forming block, a polyester dendron and a poly(ethylene) glycol (PEG) moiety. The micelles comprising amphiphilic dendron-coils are also referred to as “multivalent dendron conjugates” and “dendron-based nanomicelles (DNMs)”.

The hydrophobic core-forming block of the micelles is non-peptidyl, that is, the hydrophobic core-forming block is not a peptide. In some embodiments, a micelle comprises a single type of amphilphilic dendron-coil (i.e., the amphiphilic dendron-coils in the micelle all have the same three components.) In some embodiments, a micelle comprises more than one type of amphiphilic dendron-coil (i.e., the amphiphilic dendon-coils in the micelle vary in their three components.)

In some embodiments, the non-peptidyl, hydrophobic core-forming block of the amphiphilic dendron-coil comprises polycaprolactone (PCL), poly(lactic acid) (PLA), poly(glycolic acid) (PGA) or poly(lactic-co-glycolic acid) (PLGA). In some embodiments, the non-peptidyl, hydrophobic core-forming block is PCL. In some embodiments, the PCL is poly(c-caprolactone). In some embodiments, the non-peptidyl, hydrophobic core-forming block is PLA. In some embodiments, the non-peptidyl, hydrophobic core-forming block is PGA. In some embodiments, the non-peptidyl, hydrophobic core-forming block is PLGA. The non-peptidyl, hydrophobic core-forming block has a molecular weight including, but not limited to, a molecular weight of about 0.5 kDa to about 20 kDa. In some embodiments, the non-peptidyl, hydrophobic core-forming block is poly(c-caprolactone) with a molecular weight of about 3.5 kDa. In some embodiments, the non-peptidyl, hydrophobic core-forming block is poly(c-caprolactone) has a molecular weight of 14 kDa.

In some embodiments, the polyester dendron of the amphiphilic dendron-coil includes, but is not limited to, a generation 3 to generation 5, that is, a generation 3 (G3), a generation 4 (G4) or a generation 5 (G5), polyester dendron with either an acetylene or carboxylate core. In some embodiments, the polyester dendron is a G3 dendron. In some embodiments, the polyester dendron is a G5 dendron. In some embodiments, the polyester dendron has an acetylene core. In some embodiments, the polyester dendron is generation 3 polyester-8-hydroxyl-1-acetylene bis-MPA dendron. In some embodiments, the polyester dendron has a carboxylate core.

In some embodiments, the PEG moiety of the amphiphilic dendron-coil is a methoxy PEG (mPEG) moiety, amine-terminated PEG (PEG-NH₂) moiety, acetylated PEG (PEG-Ac) moiety, carboxylated PEG (PEG-COOH) moiety, thiol-terminated PEG (PEG-SH) moiety, N-hydroxysuccinimide-PEG (PEG-NHS) moiety, NH₂-PEG-NH₂ moiety or NH₂-PEG-COOH moiety. In some embodiments, the PEG moiety has a molecular weight including, but not limited to, a molecular weight from about 0.2 kDa to about 5 kDa. In some embodiments, the PEG moiety is an mPEG moiety. In some embodiments, the PEG moiety is an mPEG moiety with a molecular weight of about 2 kDa. In some embodiments, the PEG moiety is an mPEG moiety with a molecular weight of about 5 kDa.

In an embodiment, a polyester dendron is covalently modified with the linear hydrophobic polymer to help to facilitate chain entanglement and intramolecular interactions which aid in the self-assembly of core-shell type micelles and enable hydrophobic drug molecules to be loaded within the micelles. The PEG moieties form a hydrophilic corona with non-fouling properties and afford increased circulation half-life when the micelles are administered in vivo.

Biologically important properties such as biodegradability, circulation half-life, targetability, pharmacokinetics and drug release can be controlled by varying the three components (also referred to as the three polymer blocks) of the amphiphilic dendron-coils. Moreover, the copolymer structure is flexible and can be easily manipulated by varying the molecular weights of each component to fine-tune the hydrophilic-lipophilic balances (HLBs). For example, various embodiments employ PCL, polyester dendron, and PEG with molecular weights ranging 0.5-20 kDa, G3-G5 (approximately 0.9-3.5 kDa), and 0.2-5 kDa, respectively. The HLBs (20 M_{H}/(M_{H}+M_{L}), where M_{H} is the mass of the hydrophilic block and M_{L} is the mass of the lipophilic block) therefore widely vary from 2.22 to 19.94.

When a dendron is co-polymerized with the hydrophobic linear polymer such as polycaprolactone (PCL), poly(lactic acid) (PLA), poly(glycolic acid) (PGA), and poly(lactic-co-glycolic acid) (PLGA) in the generation of the amphiphilic dendron-coils, the cone-shaped, amphiphilic dendron-coils in turn possess advantageous structural attributes because they form self-assembled micelles, which are thermodynamically favorable and have highly packed PEG surface layers for increased blood circulation time. The thermodynamic stability in forming micelles, along with the unique architecture that is easily tunable.

The nanocarrier systems include hybrids of hyperbranched polymers and other biocompatible nanoparticles. For example, such hybrid nanoparticles include dendrimer-liposome, dendrimer-PEG-PLA, dendrimer-exosome hybrids that combine unique advantages of dendrimers (2-10 nm in diameter) and larger nanoparticles (50-200 nm).

Exemplary hybrid nanoparticles (also referred to as nanohybrids) include those described in U.S. Pat. No. 9,168,225, incorporated herein by reference for its disclosure of hybrid nanoparticles. In this embodiment, a hybrid nanoparticle is a particle in which a nanocore is surrounded or encapsulated in a matrix or shell. In other words, a smaller particle within a larger particle. In certain embodiments, the hybrid nanoparticles comprise a nanocore inside a liposome. In other embodiments, the nanocore is surrounded by a polymeric matrix or shell (e.g., a polymeric nanoparticle).

The nanocores are preferably from 1 nm to 50 nm in their greatest diameter. More preferably, the nanocores range from 1 to 40 nm in their greatest diameter, most preferably from 3 to 20 nm in their greatest diameter. The nanocores may be analyzed by dynamic light scattering and/or scanning electron microscopy to determine the size of the particles. A nanocore can have any shape and any morphology. Examples of nanocores include nanopowders, nanoclusters, nanocrystals, nanospheres, nanofibers, and nanotubes. Given its nanoscale size, the nanocore scaffold is readily excreted. Therefore, the nanocore scaffold employed need not be biodegradable, but in particular embodiments, the nanocore scaffold is biocompatible, i.e., not toxic to cells. Scaffolds are “biocompatible” if their addition to cells in vitro results in less than or equal to 30%, 20%, 10%, 5%, or 1% cell death and do not induce inflammation or other such unwanted adverse effects in vivo.

Exemplary polymeric scaffolds include, but are not limited to, a polyamide, a polysaccharide, a polyanhydride, poly-L-lysine, a polyacrylamide, a polymethacrylate, a polypeptide, a polyethylene oxide, a polyethyleneimine (PEI), or a dendrimer such as poly(amidoamine) (PAMAM) and PAMAM (ethylenediamine-EDA) dendrimers or modified versions thereof, e.g., hydroxylated, acetylated, or carboxylated versions of said polymers. Other exemplary polymeric backbones are described, e.g., in WO98/46270 (PCT/US98/07171) or WO98/47002 (PCT/US98/06963). The multivalent polymeric scaffold molecules can have a configuration selected from linear, branched, forked or star-like.

In some embodiments, at least a portion of the multivalent polymeric scaffold molecule may be hydrophobic. In some embodiments, at least a portion of the multivalent polymeric scaffold molecule may be hydrophilic. In another embodiment, a portion of the multivalent polymeric scaffold molecule may be hydrophobic, and a different portion of the molecule may be hydrophilic. In particular embodiments, the multivalent polymeric scaffold molecule is cationic. In other embodiments, the multivalent polymeric scaffold molecule is electronically neutral. In still other embodiments, the multivalent polymeric scaffold molecule is anionic. Those skilled in the art will recognize that various starting materials may be selected to obtain a multivalent polymeric scaffold molecule that exhibits the desired properties.

In one embodiment, the shell is a liposome composed of a phospholipid such as egg phosphatidylcholine, egg phosphatidylethanolamine, soy bean phosphatidylcholine, lecithin, sphingomyelin, synthetic phosphatidylcholine, lyso-phosphatidylcholine, phosphatidylglycerol, phosphatidic acid, phosphatidylethanolamine, or phosphatidylserine, wherein the phospholipid can be modified with a long-circulating agent or cryoprotectant. In another embodiment, the shell is polymeric nanoparticle composed of a polymer selected from the group of poly-(γ-L-glutamylglutamine), poly-(γ-L-aspartylglutamine), poly-L-lactic acid, poly-(lactic acid-co-glycolic acid), polyalkylcyanoacrylate, polyanhydrides, polyhydroxyacids, polypropylfumerate, polyamide, polyacetal, polyether, polyester, poly(orthoester), polycyanoacrylate, [N-(2-hydroxypropyl)methacrylamide] copolymer, polyvinyl alcohol, polyurethane, polyphosphazene, polyacrylate, polyurea, polyamine polyepsilon-caprolactone, and copolymers thereof, wherein the polymer is modified or derivatized to enhance proteolytic resistance, improve circulating half-life, reduce antigenicity, reduce immunogenicity, reduce toxicity, improve solubility, or improve thermal or mechanical stability. In particular embodiments, the shell is biodegradable. In certain embodiments the multivalent polymeric scaffold is cationic and is composed of a polyamide, a polysaccharide, a polyanhydride, poly-L-lysine, a polyacrylamide, a polymethacrylate, a polypeptide, a polyethylene oxide, a polyethyleneimine, poly(amidoamine) (PAMAM) or PAMAM (ethylenediamine-EDA).

Another hybrid nanoparticle is a dendrimer-exosome hybrid as described in U.S. application Ser. No. 16/011,922. A dendrimer-exosome hybrid is an exosome loaded with one or more nanoparticle dendrimers. As used herein, exosome refers to small vesicles having a membrane structure that are secreted from various cells. Exosomes have diameters of about 25 to about 150 nm. Exosomes may express markers such as VLA-4, CD162, CXCR4, CD9, CD63, CD81 or a combination thereof. In an embodiment, the exosome is derived from a stem cell or a tumor cell which is isolated from a subject, e.g., a human subject.

In an embodiment, the exosome is derived from a stem cell or a tumor cell which is isolated from a subject, e.g., a human subject.

Stem cells include embryonic stem cells or adult stem cells, preferably, adult stem cells. The adult stem cells may be, without being limited to, mesenchymal stem cells, human tissue-derived mesenchymal stromal cells (mesenchymal stromal cell), human tissue-derived mesenchymal stem cells, multipotent stem cells, or amniotic epithelial cells, preferably, mesenchymal stem cells. The mesenchymal stem cells may be derived from, without being limited to, the umbilical cord, umbilical cord blood, bone marrow, fat, muscle, nerve, skin, amnion, placenta, and the like.

In an embodiment, the stem cell is a mesenchymal stem cell. Mesenchymal stem cells (MSCs) can specifically target inflammatory regions that are frequently found in cancerous regions, i.e., MSC tumor-homing.

In another embodiment, the exosome is isolated from a tumor cell. Tumor cells actively produce, release, and utilize exosomes to promote tumor growth.

Exosomes can be produced by isolating tumor or stem cells from a subject, expanding the tumor or stem cells to provide an expanded cell population, culturing the expanded cell population, and isolating the exosome secreted from the expanded tumor or stem cells. The internal components can be removed from the isolated exosomes to provide so-called ghost exosomes which are essentially empty vessels for loading components such as nanoparticle dendrimers. Exosomes derived from a patient can provide a non-immunogenic nanocarrier shell to the patient, in addition to the features above, allowing an option for personalized medicine.

In order to allow for conjugation of the immune checkpoint inhibitors, in one aspect, the multivalent nanoparticles are modified by reaction with alkyl epoxides, wherein the R group of the epoxide has 1 to 30 carbon atoms. In some embodiments, the alkyl epoxides react with amino groups present on the multivalent nanoparticles to form alkylated multivalent nanoparticles.

Amine groups present on the multivalent nanoparticles provide reactive sites for a variety of amine-based conjugation reactions using coupling linkers that include, but are not limited to, dicyclohexylcarbodiimide, diisopropylcarbodiimide, N-(3-Dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptoethylamine, sulfosuccinimidyl 4-(N-maleimidomethyl)cyclohexane-1-carboxylate, succinimidyl iodoacetate, succinimidyl 3-(2-pyridyldithio)propionate. In some embodiments, reactive esters are used to link multivalent nanoparticles and other compounds via ester bonds. Examples of the reactive esters include, but are not limited to, N-hydroxysuccinimide ester, N-hydroxy sulfosuccinimide ester, N-γ-maleimidobutyryl-oxysulfosuccinimide ester, nitrophenyl ester, tetrafluoro phenyl ester, pentafluorophenyl ester, thiopyridyl ester, thionitrophenyl ester. Preferably, the reactive ester group is an N-hydroxysuccinimide ester.

The nanoparticle system comprises a plurality of conjugated ICIs. Immune checkpoints refer to a plurality of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. In an embodiment, the ICI specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137, CD155, CD266, PVR, PVR-2, CD47, CD160, NT5E, CD96, TNFRSF18, or a combination comprising one or more of the foregoing. In an embodiment, the ICI is a whole antibody, an antibody fragment, or a peptide.

Exemplary immune checkpoint inhibitors include cerniplimab-rwic, nivolumab, pembrolizumab, pidilizumab, MEDI-0680, PDR001, REGN2810, and BGB-108, AMP-224, an immunoadhesin, BMS-936559, atezolizumab, YW243.55.570, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, MSB001078C, or a combination comprising one or more of the foregoing.

In an embodiment, a PD-1 binding antagonist is an anti-PD-1 antibody (e.g., a human antibody, a humanized antibody, or a chimeric antibody). Exemplary anti-PD-1 antibodies include REGN2810 (cemiplimab), MDX 1106 (nivolumab), MK-3475 (pembrolizumab), CT-011 (pidilizumab), MEDI-0680 (AMP-514), PDR001, and BGB-108 (Tislelizumab). In an embodiment, the PD-1 binding molecule is an immunoadhesin (e.g., an immunoadhesin comprising an extracellular or PD-1 binding portion of PD-L1 or PD-L2 fused to a constant region (e.g., an Fc region of an immunoglobulin sequence). In an embodiment, the PD-1 binding molecule is AMP-224. AMP-224, also known as B7-DCIg, is a PD-L2-Fc fusion soluble receptor described in WO2010/027827 and WO2011/066342.

MDX-1106, also known as MDX-1106-04, ONO-4538, BMS-936558, or nivolumab, is an anti-PD-1 antibody described in WO2006/121168. MK-3475, also known as lambrolizumab (pembrolizumab), is an anti-PD-1 antibody described in WO2009/114335. CT-011, also known as hBAT, hBAT-1 or pidilizumab, is an anti-PD-1 antibody described in WO2009/101611.

In an embodiment, the PD-L1 binding antagonist is anti-PD-L1 antibody. Exemplary anti-PD-L1 antibodies include MPDL3280A (atezolizumab), YW243.55.570, MDX-1105, MEDI4736 (durvalumab), and MSB0010718C (avelumab). Antibody YW243.55.570 is an anti-PD-L1 described in WO 2010/077634. MDX-1105, also known as BMS-936559, is an anti-PD-L1 antibody described in WO2007/005874. MEDI4736 is an anti-PD-L1 monoclonal antibody described in WO2011/066389 and US2013/034559.

Additional ICIs include ipilimumab (anti-CTLA-4), tremelimumab (anti-CTLA-4), BMS-986016 (anti-LAG-3), urelumab (anti-4-1BB), MSB001078C (anti-4-1BB), TRX51 (anti-GITR), dacetuzumab (anti-CD40), lucatumumab (anti-CD40), SEA-CD40 (anti-CD40), CP-870,893 (anti-CD40), MED16469 (OX40), and MOXR0916 (OX40).

The large number of end groups on the multivalent nanoparticle core allows for conjugation of a wide variety of molecules in addition to the ICIs. The multivalent nanoparticle core can be associated with, e.g., complexed or conjugated with, one or more of a therapeutic, prophylactic or diagnostic agent. Diagnostic agents include imaging agents.

In one aspect, the therapeutic agent is a chemotherapeutic agent. Chemotherapeutic agents include, but are not limited to, the following classes: alkylating agents, antimetabolites, anthracyclines, plant alkaloids, topoisomerase inhibitors, monoclonal antibodies, and other anti-tumor agents. In addition to the chemotherapeutic drugs described above, namely doxorubicin, paclitaxel, other suitable chemotherapy drugs include tyrosine kinase inhibitor imatinib mesylate (Gleeve® or Glivec®), cisplatin, carboplatin, oxaliplatin, mechloethamine, cyclophosphamide, chlorambucil, azathioprine, mercaptopurine, pyrimidine, vincristine, vinblastine, vinorelbine, vindesine, podophyllotoxin (L01CB), etoposide, docetaxel, topoisomerase inhibitors (L01CB and L01XX) irinotecan, topotecan, amsacrine, etoposide, etoposide phosphate, teniposide, dactinomycin, lonidamine, and monoclonal antibodies, such as trastuzumab (Herceptin®), cetuximab, bevacizumab and rituximab (Rituxan®), among others.

Other examples of therapeutic agents include, but are not limited to, antimicrobial agents, analgesics, anti-inflammatory agents, and others. Antibiotics can be incorporated into the particle, such as vancomycin, which is frequently used to treat infections, including those due to methicillin resistant staph aureus (MRSA). The particle optionally includes cyclosporin, a lipophilic drug that is an immunosuppressant agent, widely used post-allogeneic organ transplant to reduce the activity of the patient's immune system and the risk of organ rejection (marketed by Novartis under the brand names Sandimmune® and Neoral®). Particles comprising cyclosporine can be used in topical emulsions for treating keratoconjunctivitis sicca, as well. In this regard, particles with multifunctional surface domains incorporating such drugs can be designed to deliver equivalent dosages of the various drugs directly to the cancer cells, thus potentially minimizing the amount delivered generally to the patient and minimizing collateral damage to other tissues.

Therapeutic agents also include therapeutic nucleic acids such as gene-silencing agents, gene-regulating agents, antisense agents, peptide nucleic acid agents, ribozyme agents, RNA agents, and DNA agents. Nucleic acid therapeutic agents include single stranded or double-stranded RNA or DNA, specifically RNA, such as triplex oligonucleotides, ribozymes, aptamers, small interfering RNA including siRNA (short interfering RNA) and shRNA (short hairpin RNA), antisense RNA, microRNAs (miRNAs), or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence.

Diagnostic agents are agents that enable the detection or imaging of a tissue or disease. Examples of diagnostic agents include, but are not limited to, radiolabels, fluorophores and dyes.

Imaging agent refers to a label that is attached to the random copolymer of the present invention for imaging a tumor, organ, or tissue in a subject. Examples of imaging agents include, without limitation, radionuclides, fluorophores such as fluorescein, rhodamine, isothiocyanates (TRITC, FITC), Texas Red, Cy2, Cy3, Cy5, APC, and the AlexaFluor® (Invitrogen, Carlsbad, Calif.) range of fluorophores, antibodies, gadolinium, gold, nanomaterials, horseradish peroxidase, alkaline phosphatase, derivatives thereof, and mixtures thereof.

Radiolabel refers to a nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive sub stance.

Administration of a prophylactic agent can occur prior to the manifestation of symptoms characteristic of the disease or disorder, such that the disease or disorder is prevented or, alternatively, delayed in its progression.

Therapeutic molecules, diagnostic agents, and prophylactic agents may be combined with multivalent nanoparticle core via chemical conjugation, physical encapsulation, and/or electrostatic interaction methods.

Also included are pharmaceutical compositions comprising the nanoparticle system described herein. Pharmaceutical compositions may further comprise the therapeutic, prophylactic or diagnostic agent as described above.

As used herein, “pharmaceutical composition” means therapeutically effective amounts of the nanoparticles together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the skin, the drug may be made up into a cream, lotion or ointment. Cream or ointment formulations which may be used for the drug are conventional formulations well known in the art. Topical administration includes transdermal formulations such as patches.

For topical application to the eye, the inhibitor may be made up into a solution or suspension in a suitable sterile aqueous or non-aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anesthetics, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

In an aspect, a method of making a nanoparticle system comprises contacting the multivalent nanoparticle cores comprising multiple reactive end groups with a composition comprising immune checkpoint inhibitors under conditions sufficient to conjugate a plurality of immune checkpoint inhibitors to the multivalent nanoparticle cores and provide the nanoparticle system. Exemplary end groups include coupling linkers and reactive epoxides, such as dicyclohexylcarbodiimide, diisopropylcarbodiimide, N-(3-dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptoethylamine, sulfosuccinimidyl 4-(N-maleimidomethyl)cyclohexane-1-carboxylate, succinimidyl iodoacetate, succinimidyl 3-(2-pyridyldithio)propionate, N-hydroxysuccinimide ester, N-hydroxy sulfosuccinimide ester, N-γ-mal eimidobutyryl-oxysulfosuccinimide ester, nitrophenyl ester, tetrafluoro phenyl ester, pentafluorophenyl ester, thiopyridyl ester, thionitrophenyl ester, and combinations comprising at least one of the foregoing.

In an embodiment, the multivalent nanoparticle cores comprise two or more different types of reactive end groups to enhance the reactivity and/or specificity of the cores.

In another embodiment, an immunotherapy method comprises administering to the subject, e.g., a human subject, a nanoparticle system as described herein. Exemplary human subjects include cancer patients and patients with immune disorders such as multiple sclerosis and rheumatoid arthritis. The nanoparticles can target the immune system by interacting with T cells, cancer cells and/or antigen presenting cells.

The compositions and methods described herein are applicable to all cancers including solid tumor cancers, e.g., those of the breast, prostate, ovaries, lungs and brain, and liquid cancers such as leukemias and lymphomas.

The methods described herein can be further combined with additional cancer therapies such as radiation therapy, chemotherapy, surgery, and combinations thereof.

The invention is further illustrated by the following non-limiting examples.

### EXAMPLES

### Example 1: Synthesis of a Dendrimer-ICI Antibody Conjugate

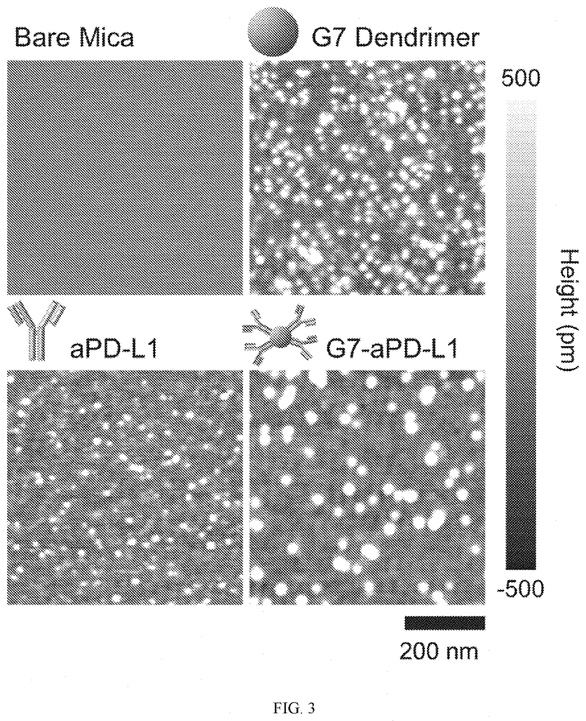

FIG. 2 demonstrates an exemplary synthesis process of polymer-inhibitor conjugates including a generation 7(G7) poly(amidoamine)(PAMAM) dendrimer and four PD-L1 antibodies (G7-aPD-L1 conjugates). A variety of conjugation chemistries could be employed to form different polymer-inhibitor conjugates. In this example, G7 PAMAM dendrimers were labelled with Alexa Fluor® 647, followed by partial acetylation with acetic anhydride. Approximately 90% of amine terminal groups were acetylated in order to reduce the steric hinderance. The remaining amine terminal groups were then carboxylated by the reaction with succinic anhydride. The carboxyl end groups on dendrimers were conjugated with amine groups of aPD-L1 using the EDC/NHS chemistry. Approximately 3.9±0.6 antibodies were conjugated to each dendrimer.

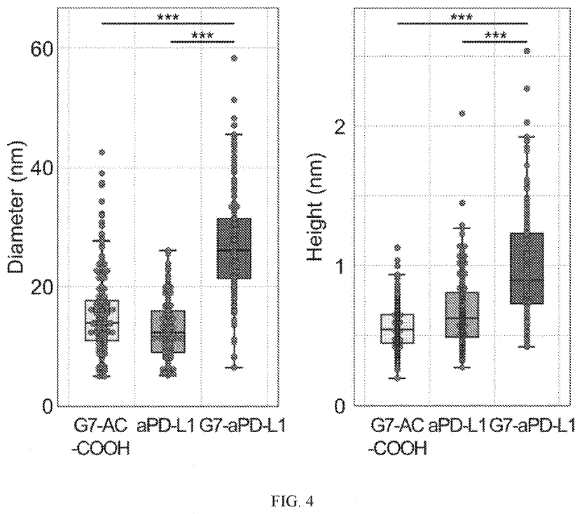

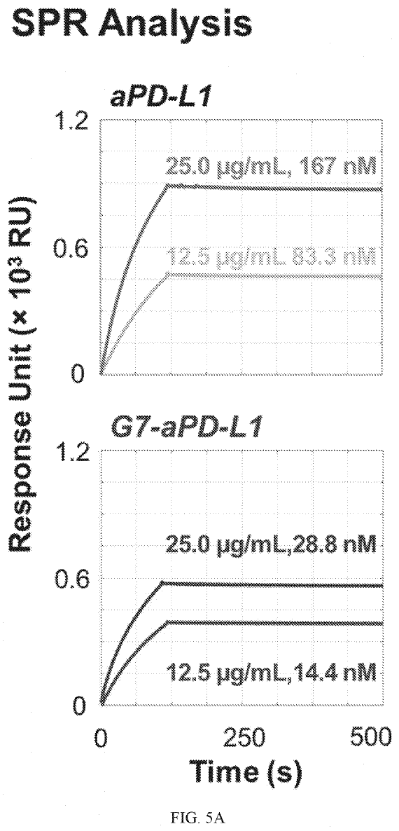

FIGS. 3 and 4 show characterization of the dendrimer conjugates using AFM, confirming the successful conjugation between G7 dendrimers and antibodies. AFM images demonstrate a significant increase in both lateral diameter (D) and height (h) of the G7-Ab conjugates (D=27.4±8.9 nm/h=9.8±3.9 Å) compared to free antibodies (D=12.7±4.4 nm; p<0.001/h=6.7±2.5 Å; p<0.001) and G7 PAMAM dendrimers (D=16.3±7.3 nm; p<0.001/h=5.6±1.6 Å; p<0.001).

### Example 2: Confirmation of Enhanced Binding Kinetics

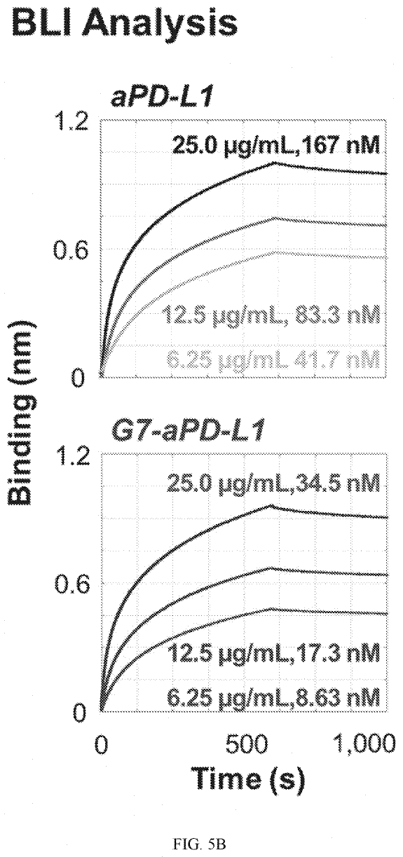

FIG. 5 shows the enhancement in binding affinity of G7-aPD-L1, using (5A) surface plasmon resonance (SPR), (5B) biolayer interferometry (BLI), and (5C) atomic force microscopy (AFM): (5A, B) G7-aPD-L1 conjugates exhibited up to two orders of magnitude lower dissociation constant (K_{D}) compared to free aPD-L1; (5C) G7-aPD-L1 conjugates tended to show higher rupture force with multiple rupture events compared to aPD-L1 as shown (Left). Histogram of rupture forces as different loading rates were fitted into double Gaussian model (Middle). These were translated into Bell-Evans model to obtain dissociation rate (Right). G7-aPD-L1 conjugates demonstrated an order of magnitude enhanced off-rate kinetics compared to aPD-L1. (5D). In summary, G7-aPD-L1 exhibited significantly higher binding kinetics than aPD-L1.

In the SPR method, carboxymethylated dextran was covalently attached to a gold surface. Polarized light strikes the electrically conducting surface at the interface providing reflected electron charge density waves. The angle of the reflected light changes as molecules bind and dissociate at the surface, and the interaction profile is recorded in a sensorgram. The BLI method is a label-free biosensor method that can take real-time measurements of molecular interactions. It detects changes in the interference pattern of white light reflected back from the surface of fiber optic biosensors. The x-axis is time (s) and y-axis is in nm. Since there is no flow, the raw data shows changes of wavelength in BLI interference peaks (nm) in binding to surface of biosensor which is a function of changes to average optical thickness. For association, the wavelength shift to the right in real time. For dissociation, the wavelength shifts back to its original position.

Both SPR and BLI results demonstrate that the dissociation constant (K_{D}) of G7-aPD-L1 conjugates were lower than that of free antibodies by up to two orders of magnitude, indicating that the conjugates are more strongly bound with the target protein.

TABLE 1
SPR results

| | Free Ab | Conjugate |
|---|---|---|
| kₐ (1/Ms) | 7.68 × 10⁴ | 5.53 × 10⁶ |
| k_{d} (1/s) | 2.83 × 10⁻⁵ | 2.51 × 10⁻⁵ |
| K_{D} (M) | 3.69 × 10⁻¹⁰ | 4.54 × 10⁻¹² |

• K_{D} value measured at the concentration of 25 μg/mL (166.7 nM for free Abs and 34.5 nM for conjugate)

TABLE 2
BLI results

| | Free Ab | Conjugate |
|---|---|---|
| kₐ (1/Ms) | 2.38 × 10⁵ | 1.18 × 10⁶ |
| k_{d} (1/s) | 2.75 × 10⁻⁴ | 6.79 × 10⁻⁵ |
| K_{D} (M) | 1.16 × 10⁻⁹ | 6.16 × 10⁻¹¹ |

• K_{D} value measured at the concentration of 25 μg/mL (166.7 nM for free Abs and 34.5 nM for conjugate)

### Example 3: Target Specificity and Enhanced Binding Kinetics of G7-aPD-L1

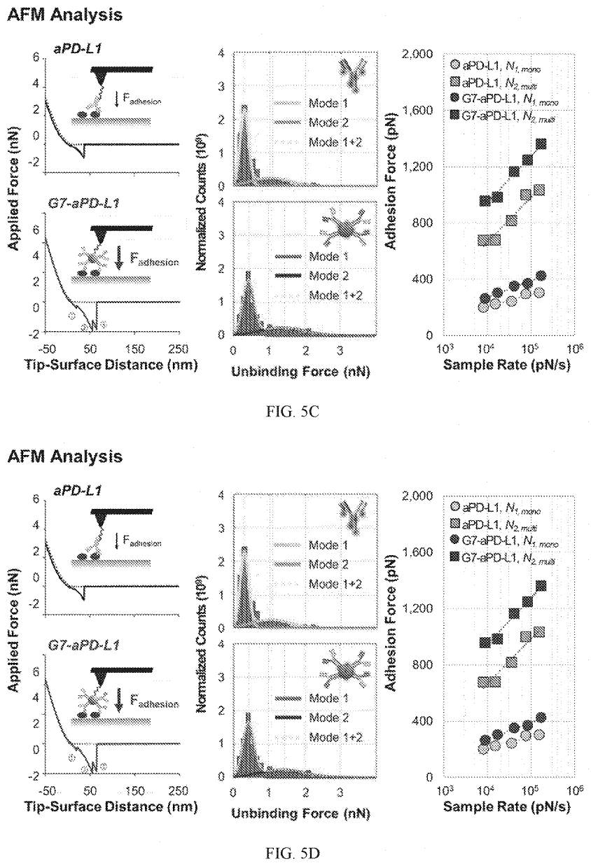

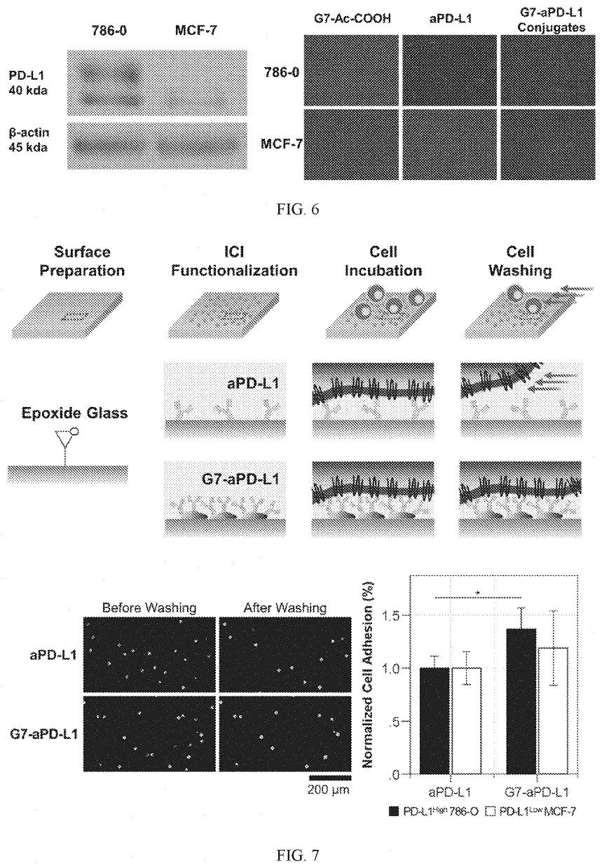

Target specificity and enhanced binding kinetics of G7-aPD-L1 compared to aPD-L1 was confirmed in vitro. FIG. 6 shows PD-L1 expressions of 786-O (PD-L1^{High}) and MCF-7 (PD-L1^{LOW}) cell lines was quantified by western blot. Expressions of both aPD-L1 and G7-aPD-L1 were significantly higher in 786-O cell line compared to MCF-7. FIG. 7 shows the enhanced binding kinetics were verified in vitro via cell retention assay. In FIG. 7, cancer cells were suspended on the surface functionalized with either G7-aPD-L1 conjugates or aPD-L1. PD-L1^{High} cancer cells showed 1.4-fold (p<0.05) enhanced retention on the surface covered with the G7-aPD-L1 conjugates at a shear rate of 25 s⁻¹, compared to that with free antibodies.

### Example 4: Enhanced Blockade of PD-1/PD-L1 Interaction Via G7-aPD-L1 Conjugates

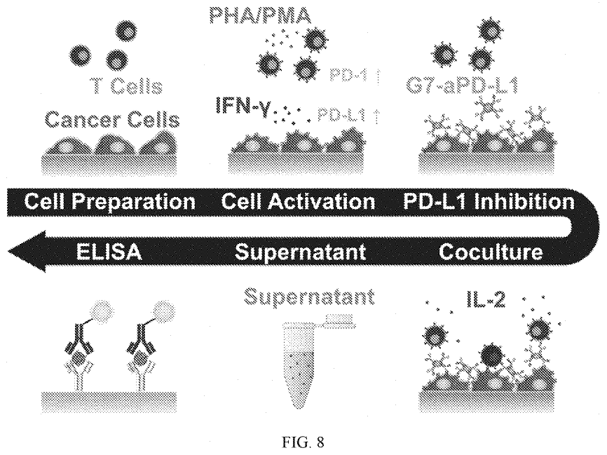

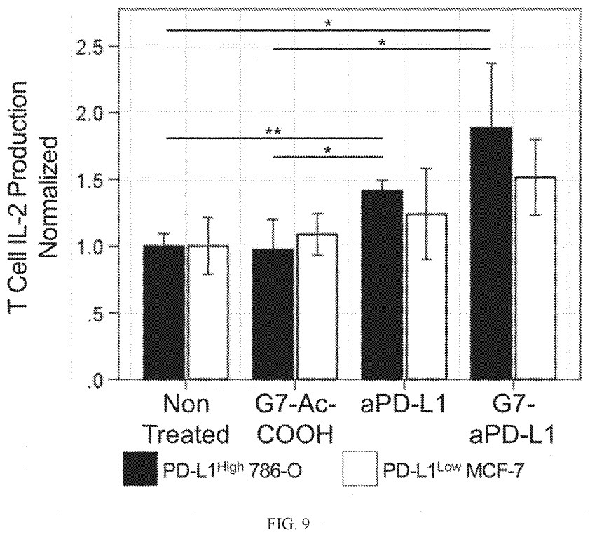

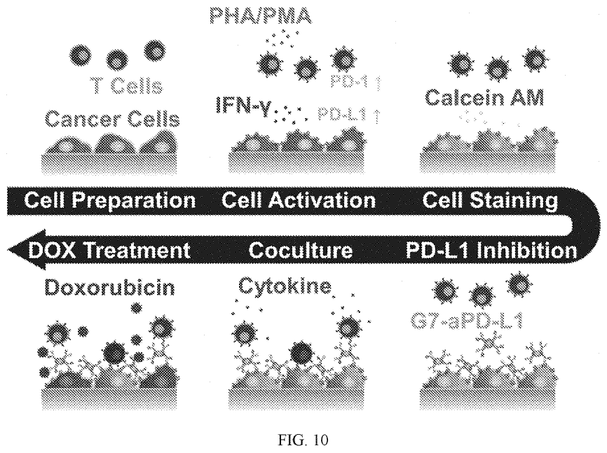

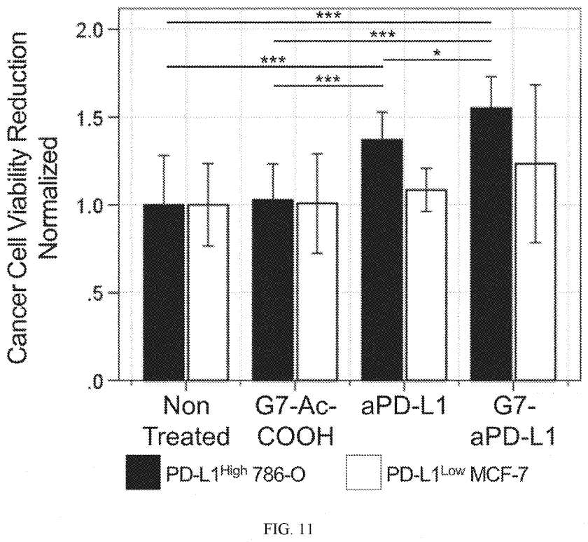

Enhanced blockade of PD-1/PD-L1 interaction via G7-aPD-L1 conjugates was confirmed in vitro by (FIGS. 8 and 9) assessing Jurkat T cell production of IL-2 and (FIGS. 10 and 11) measuring chemo-sensitivity. The blockade of the PD-1/PD-L1 pathway via G7-aPD-L1 resulted in 1.9-fold enhancement in T cell IL-2 production (p=0.036) and 9% reduction in chemoresistance of 786-0 cells to doxorubicin (p=0.002), compared to non-ICI treated cells. The results were superior to free antibodies which only showed 1.4-fold enhancement (p=0.004) in T cell IL-2 production and 5% reduction in cancer cell chemoresistance (p=0.020). Note that non-targeted dendrimers have no significant effect in blocking PD-1/PD-L1 interaction.

### Example 5: Mouse Study

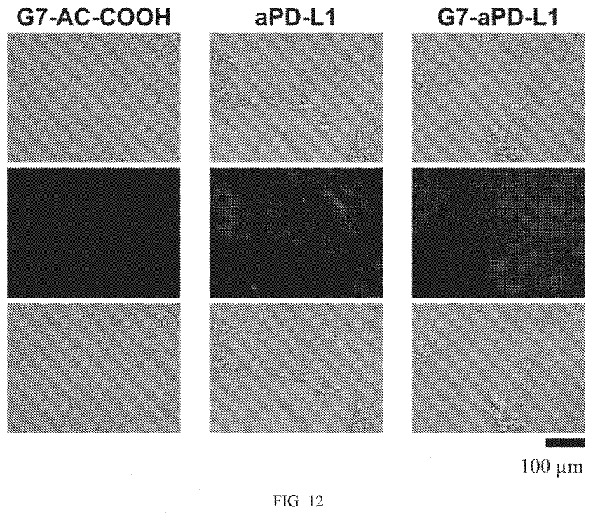

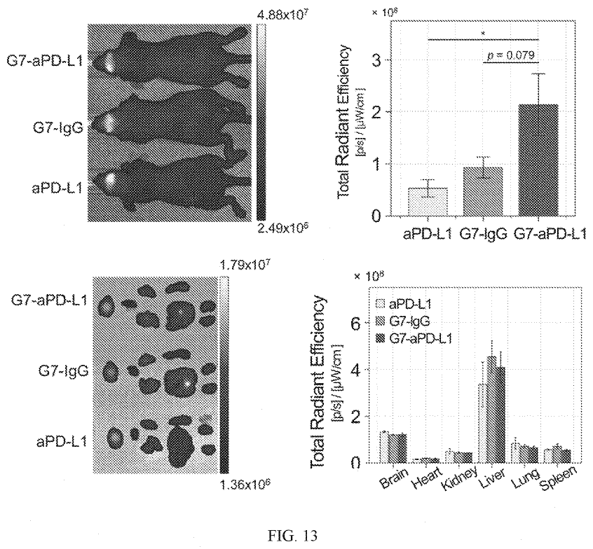

Enhanced blockade of PD-1/PD-L1 interaction via G7-aPD-L1 was further confirmed by in vivo mouse model study as shown in FIGS. 12 and 13. Both aPD-L1 fluorophore-labelled aPD-L1 and G7-aPD-L1 were highly expressed in MOC1 cells at the concentration of 67 nM. However, the expressions of both inhibitors were significantly reduced when the PD-L1 ligands were blocked by pre-treating the cells with 670 nM of nonfluorescent aPD-L1. FIG. 13 shows enhanced targeting of G7-aPD-L1 using in vivo mouse model. Experiments were conducted using 4- to 6-week-old female C57BL/6 mice which were obtained from the Envigo Laboratories (Indianapolis, Ind.). All animal procedures and maintenance were conducted in accordance with the institutional guidelines of the University of Wisconsin. To establish an in vivo mouse tumor model, approximately 5×10⁵ MOC1 cells were injected to the mice. Once the tumor reached 300 mm³, 50 μL of either G7-aPD-L1 or aPD-L1 was injected through the tail vein of the tumor-bearing mouse at the concentration of 128 nM. In vivo imaging system (IVIS) analysis reveals approximately 2-fold enhancement of G7-aPD-L1 for targeting the tumor, compared to aPD-L1.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A nanoparticle system comprising
a multivalent nanoparticle core comprising a plurality of immune checkpoint inhibitors conjugated thereto.

2. The nanoparticle system of claim 1, wherein the multivalent nanoparticle core comprises a hyperbranched polymer, a dendrimer, a dendron, a hybrid nanoparticle, or a micelle.

3. The nanoparticle system of claim 2, wherein the micelle comprises an amphiphilic dendron-coil.

4. The nanoparticle system of claim 2, wherein the hybrid nanoparticle comprises a dendrimer-exosome hybrid.

5. The nanoparticle system of claim 2, wherein the hybrid nanoparticle comprises a multivalent polymeric scaffold nanoparticle core with the immune checkpoint inhibitor covalently attached thereto; and an outer shell encapsulating the polymeric scaffold nanoparticle core, wherein the outer shell comprises a liposome or a polymeric shell.

6. The nanoparticle system of claim 2, wherein the dendrimer is a poly(amido-amine) (PAMAM) dendrimer, a polyester dendrimer, a polypropyleneimine (PPI) dendrimer, a diaminobutane amine polypropylenimine tetramine (DAB-Am 4) dendrimer, a polypropylamine (POPAM) dendimer, a polylysine dendrimer, a polyester dendrimer, an iptycene dendrimer, a aliphatic poly(ether) dendrimer, an aromatic polyether dendrimer, or a combination thereof.

7. The nanoparticle system of claim 2, wherein the dendrimer is a PAMAM dendrimer.

8. The nanoparticle system of claim 1, wherein the immune checkpoint inhibitor specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137, CD155, CD266, PVR, PVR-2, CD47, CD160, NT5E, CD96, or TNFRSF18.

9. The nanoparticle system of claim 8, wherein the immune checkpoint inhibitor is a whole antibody, an antibody fragment, or a peptide.

10. The nanoparticle system of claim 8, wherein the immune checkpoint inhibitor comprises cemiplimab-rwlc, nivolumab, pembrolizumab, pidilizumab, MEDI-0680, PDR001, REGN2810, and BGB-108, AMP-224, an immunoadhesin, BMS-936559, atezolizumab, YW243.55.S70, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, or MSB001078C.

11. The nanoparticle system of claim 1, wherein the nanoparticle system is further associated with a therapeutic, prophylactic or diagnostic agent.

12. The nanoparticle system of claim 11, wherein the therapeutic agent is a chemotherapeutic agent or a therapeutic nucleic acid.

13. The nanoparticle system of claim 11, wherein the diagnostic agent is an imaging agent.

14. A pharmaceutical composition comprising the nanoparticle system claim 1 and a pharmaceutically acceptable excipient.

15. The pharmaceutical composition of claim 14, further comprising a therapeutic, prophylactic or diagnostic agent.

16. A method of making a nanoparticle system, comprising
contacting multivalent nanoparticle cores comprising multiple reactive end groups with a composition comprising one or more immune checkpoint inhibitors under conditions sufficient to conjugate a plurality of the immune checkpoint inhibitors to the multivalent nanoparticle cores and provide the nanoparticle system.

17. The method of claim 16, wherein the reactive end groups comprise dicyclohexylcarbodiimide, diisopropylcarbodiimide, N-(3-Dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptoethylamine, sulfosuccinimidyl 4-(N-maleimidomethyl)cyclohexane-1-carboxylate, succinimidyl iodoacetate, succinimidyl 3-(2-pyridyldithio)propionate, N-hydroxysuccinimide ester, N-hydroxy sulfosuccinimide ester, N-γ-maleimidobutyryl-oxysulfosuccinimide ester, nitrophenyl ester, tetrafluoro phenyl ester, pentafluorophenyl ester, thiopyridyl ester, thionitrophenyl ester, or a combination thereof.

18. The method of claim 16, wherein the multivalent nanoparticle cores comprise two or more different reactive end groups.

19. The method of claim 16, further comprising contacting the multivalent nanoparticle cores comprising multiple reactive end groups with a therapeutic, prophylactic or diagnostic agent.

20. The method of claim 16, wherein the multivalent nanoparticle core comprises a hyperbranched polymer, a dendrimer, a dendron, a hybrid nanoparticle, or a micelle.

21. The method of claim 20, wherein the micelle comprises an amphiphilic dendron-coil.

22. The method of claim 20, wherein the hybrid nanoparticle comprises a dendrimer-exosome hybrid.

23. The method of claim 20, wherein the hybrid nanoparticle comprises a multivalent polymeric scaffold nanoparticle core with the immune checkpoint inhibitor covalently attached thereto; and an outer shell encapsulating the polymeric scaffold nanoparticle core, wherein the outer shell comprises a liposome or a polymeric shell.

24. The method of claim 20, wherein the dendrimer is a poly(amido-amine) (PAMAM) dendrimer, a polyester dendrimer, a polypropyleneimine (PPI) dendrimer, a diaminobutane amine polypropylenimine tetramine (DAB-Am 4) dendrimer, a polypropylamine (POPAM) dendimer, a polylysine dendrimer, a polyester dendrimer, an iptycene dendrimer, a aliphatic poly(ether) dendrimer, an aromatic polyether dendrimer, or a combination thereof.

25. The method of claim 20, wherein the dendrimer is a PAMAM dendrimer.

26. The method of claim 20, wherein the immune checkpoint inhibitor specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137, CD155, CD266, PVR, PVR-2, CD47, CD160, NT5E, CD96, or TNFRSF18.

27. The method of claim 20, wherein the immune checkpoint inhibitor is a whole antibody, an antibody fragment, or a peptide.

28. The method of claim 20, wherein the immune checkpoint inhibitor comprises cemiplimab-rwlc, nivolumab, pembrolizumab, pidilizumab, MEDI-0680, PDR001, REGN2810, and BGB-108, AMP-224, an immunoadhesin, BMS-936559, atezolizumab, YW243.55.S70, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, or MSB001078C.

29. An immunotherapy method comprising administering to a subject in need thereof the nanoparticle system of claim 1.

30. The immunotherapy method of claim 29, wherein the subject is a human cancer patient or a human patient with an immune disorder.

31. The immunotherapy method of claim 29, further comprising administering radiation therapy, chemotherapy, surgery, or a combination thereof.
